(19) United States
(12) Patent Application Publication
(10) Pub. No.: US 2025/0266126 A1
(43) Pub. Date: Aug. 21, 2025

(54) MULTI-STAGE SINGLE NUCLEOTIDE POLYMORPHISM BASED IDENTITY VERIFICATION FOR GENETIC FILES

(71) Applicant: Helix, Inc., San Mateo, CA (US)

(72) Inventors: Dana Wyman, San Diego, CA (US); Akshay Jain, Mountain View, CA (US)

(21) Appl. No.: 18/581,913

(22) Filed: Feb. 20, 2024

(51) Int. Cl. G16B 20/20; G16B 40/00

(52) U.S. Cl. CPC G16B 20/20; G16B 40/00

(57) ABSTRACT

Various embodiments disclosed relate to a method of quality control for genetic samples. A method may receive a first variant call file, comparing the first variant call file to a second variant call file across a predetermined set of single nucleotide polymorphisms; and determining whether the first variant call file and the second variant call file originate from the same individual by determining whether at least a threshold amount of single nucleotide polymorphisms within the predetermined set match between the first variant call file and the second variant call file.

### TECHNICAL FIELD

The subject matter disclosed herein generally relates to genetic information files and to quality control of those files.

### BACKGROUND

Biological samples can be analyzed for genetic data. For example, assays or assay panels can be designed to collect genetic material from an individual to screen that individual for various genetic conditions or analyses. For example, an assay may include probes directed to various genetic material of interest. Once the targeted genetic material is collected through the assay probes, such genetic material can be sequenced. The sequencing data can then be analyzed in bioinformatic processes to provide valuable biological information.

Variant call format (VCF) files are a standard text file format used in bioinformatics to store genetic sequence information. Often the content of VCFs is strictly limited to genetic sequence information. This may make it difficult to determine the origin of VCFs.

### SUMMARY OF THE DISCLOSURE

In some aspects, the techniques described herein relate to a quality control method including: receiving a first variant call file; comparing the first variant call file to a second variant call file across a predetermined set of single nucleotide polymorphisms; and determining whether the first variant call file and the second variant call file originate from the same individual by determining whether at least a threshold amount of single nucleotide polymorphisms within the predetermined set match between the first variant call file and the second variant call file.

In some aspects, the techniques described herein relate to a quality control method including: receiving a first variant call file; receiving a second variant call file; comparing the first variant call file to the second variant call file at a predetermined number of single nucleotide polymorphisms; and determining whether the first variant call file and the second variant call file are from the same individual based on comparing at the predetermined single nucleotide polymorphisms.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A depicts a diagram of an example bioinformatics analysis system.

FIG. 1B is a flow chart illustrating a method of using a bioinformatics analysis system.

FIG. 2A is a flow chart illustrating a method of intaking and preparing a genetic sample in a wet laboratory for bioinformatic analysis in an example.

FIG. 2B is a flow chart illustrating a method of intaking a genetic sample in a wet laboratory for bioinformatic analysis in an example.

FIG. 2C is a flow chart illustrating a method of preparing a genetic sample in a wet laboratory for bioinformatic analysis in an example.

FIG. 3A is a flow chart depicting a method of quality control in an example.

FIG. 3B is a flow chart depicting a method of quality control in an example.

FIG. 4 is a schematic diagram of a cloud computing system in an example.

### DETAILED DESCRIPTION

Discussed herein is a method of quality control by SNP-based fingerprinting of genetic data. The methods allow for verification of genetic information to be performed without relying on personally identifiable information (PII). The method can be reliable and efficient.

The method itself can include receiving genetic data (e.g., FASTQ-format) to an analytical tool. To ensure privacy, the received genetic data does not include identifying information. The analytical tool processes the genetic data to produce two items: a Compressed Reference-oriented Alignment Map (CRAM) file (alignment information) and a variant call file (VCF).

The produced VCF is then re-associated with the correct individual and identifying information. This is done by comparing the produced VCF with the contents of a premade VCF (such as made by an in-house analytical tool which performs a different analysis such as interpretation of exome data).

The produced VCF and the premade VCF are compared at a predefined set of single nucleotide polymorphisms (SNPs). If the produced VCF matches the premade VCF at a statistically significant amount of the predetermined SNPs, the identity can be verified. Thus, the comparison can help determine whether this is the same individual.

In bioinformatics and genomics, multiple analytical tools may be used to process genetic sequencing data in various incoming formats. For example, multiple data processing software programs that review genetic data can be used. Such analytical tools can process sequencing data in a first file format, such as a Binary Alignment Map (BAM) file or a Compressed Reference-oriented Alignment Map (CRAM) file.

The analytical tool may process such files and output data in a new file in a second file format, such as a variant call format (VCF). During this process, it is not always possible to form a chain of custody of this genetic data during the transfer from the first file format to the second file format. Thus, confusion can arise regarding which individual the genetic material refers to.

Other methods for tracking the identity corresponding to genetic data can include adding metadata to indicate the identity. However, this can result in complications related to patient privacy, such as if the second format is unintentionally exposed. Moreover, this can increase the size of the second format file, and increase processing burdens. When an analytical tool is provided by a third party, and metadata is included, this can be unacceptable, as the metadata will identify the individuals linked to the genetic data to the third party.

When parallel processing or batch processing are performed, the ability to audit output of that tool may be concerning when confirming whether the genetic information corresponds to the person indicated in the metadata.

The methods discussed herein can provide several advantages, some of which are unexpected. Discussed herein, the process of performing Single Nucleotide Polymorphism (SNP)-based verification between sets of sequencing data can be used. For SNP-based verification, not all genomic information needed may be available to perform validation. An advantage over other techniques includes the ability to enable communications with a third party or otherwise non-private analytical tool without compromising individual privacy.

### Definitions

As used herein, “accession”, or “accessioning” refers to receiving and preparing a sample for later laboratory processes.

As used herein, “amplifying” refers to the production of multiple copies of a sequence of nucleic acid or other genetic material, such as RNA or DNA.

As used herein, “bioinformatics” refers to the science of collecting complex biological data such as genetic codes.

As used herein, “biological sample”, or “sample” refers to a specimen from a patient or other organism, such as for bioinformatic research.

As used herein, “calling” an allele can include identifying one or more alleles, such as alternative alleles or mutations, at a particular locus of sequenced genetic material.

As used herein, “contamination” refers to a sample that is impure, polluted, or unsuitable for biological analysis and research.

As used herein, “genetic material” refers to a fragment, molecule, or a group of nucleic acids, such as DNA or RNA, or other genetic material, such as mitochondrial genetic material.

As used herein, “haploid” refers to genetic material having a single set of unpaired chromosomes.

As used herein, “locus” or “loci” refers to the position of a gene or mutation on a chromosome or on a fragment of genetic material.

As used herein, “mutation” refers to a changed structure of a gene that results in a variant form of the gene (e.g., with respect to a reference genome).

As used herein, “read” or “read pair” refers to data that defines a DNA or RNA sequence from one fragment or small section of genetic material.

As used herein, “recombinant” refers to genetic material formed by recombination (e.g., recombination of genetic material from two or more different variants).

As used herein, “sequencing” refers to a process of determining the nucleic acid sequence, the order of nucleotides in genetic material.

As used herein, “variant” or “genetic variant” refers to a subtype of a microorganism that is genetically distinct from other subtypes.

As used herein “walking” a sequence of genetic material can include reviewing and reading a sequenced portion of genetic material from the 4′ end towards the 3′ end to determine whether particular genetic markers, such as mutations, are present.

### Bioinformatic Analysis System and Methods

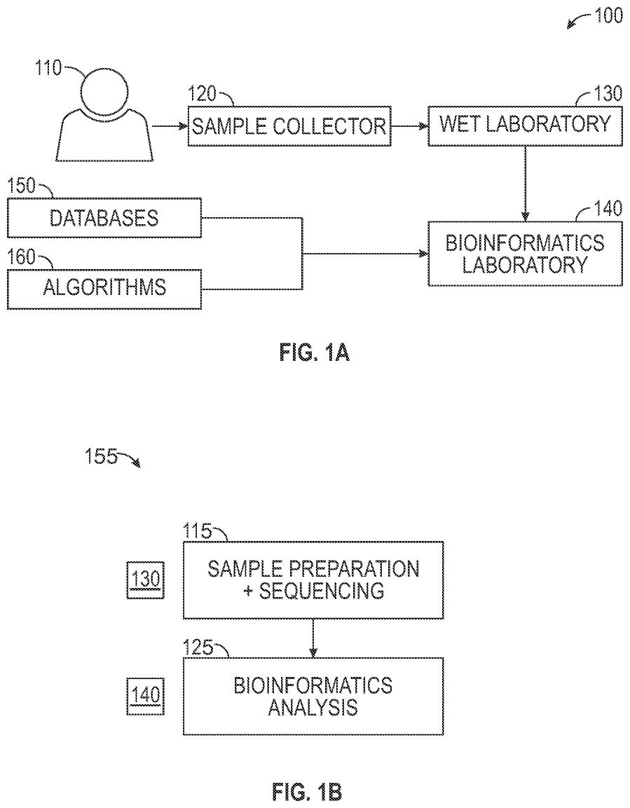

FIG. 1A depicts a diagram of an example bioinformatics analysis system 100, while FIG. 1B is a flow chart illustrating a method 155 of using such a bioinformatics analysis system 100.

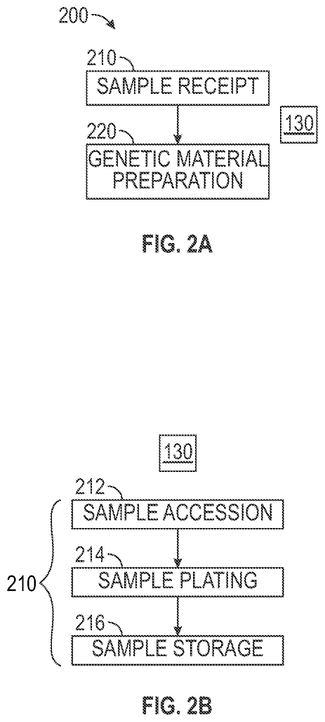

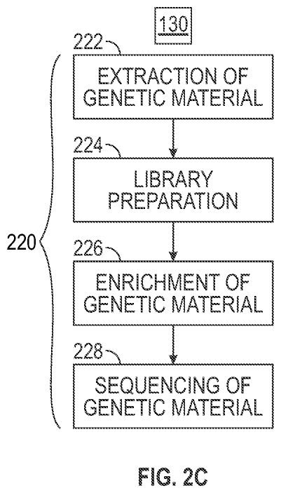

The system 100 can include both physical or “wet” laboratory components, and bioinformatics components. For example, the system 100 can interact with patients 110, from whom biological samples can be collected. The system 100 can further interact with sample collectors 120, which may be, for example, doctors, pharmacies, or other appropriate places or entities that can acquire patient samples. The system 100 includes a wet laboratory 130 which is positioned to receive the biological samples and process those samples to produce sequenced genetic material for analysis, such as at step 115 of method 155. These methods of sample receipt, handling (e.g., accession), and sequencing, are discussed in detail below with reference to FIGS. 2A to 2C.

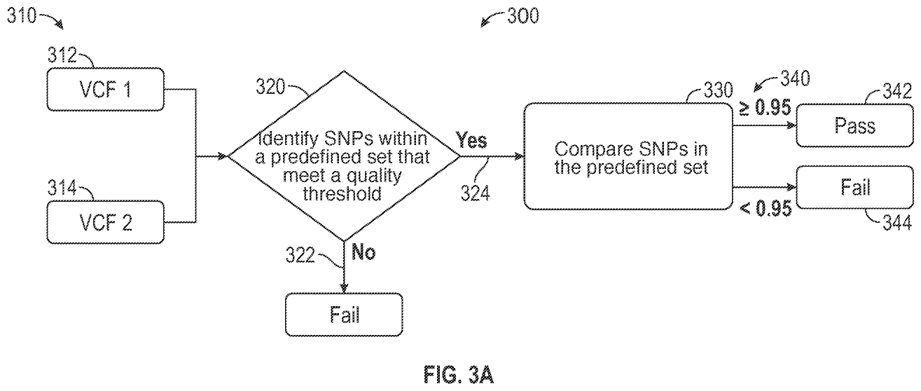

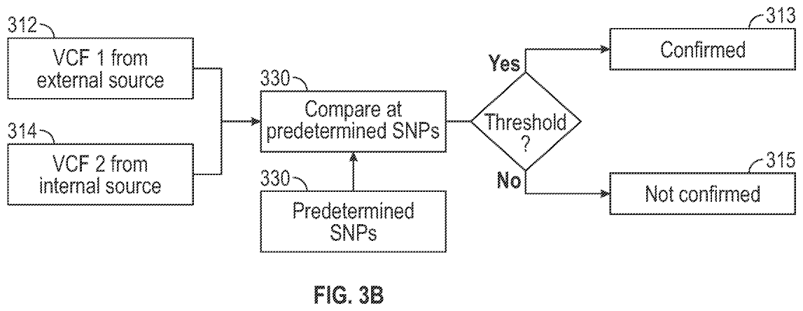

The system 100 can additionally include data driven components, such as databases 150 and algorithms 160 or other programs that support the bioinformatic laboratory 140 used to analyze genetic information. These data driven components can be used to do bioinformatic analysis (step 125 in method 155). Specific examples of such bioinformatic analysis are discussed in detail below with reference to FIGS. 3A to 3B.

### Sample Processing Methodology

Before bioinformatic analysis, biological samples are collected and sequenced through physical components of the system 100, such as through a wet laboratory 130. Methods of receiving and processing such samples are summarized in FIGS. 2A to 2C. FIG. 2A is a flow chart illustrating a method 200 of intaking and preparing a genetic sample in a wet laboratory for bioinformatic analysis. The method 200 can include two primary portions: receiving the samples (step 210) and preparing genetic material (step 220). FIG. 2B illustrates portions of step 210, including a method of intaking a genetic sample in a wet laboratory for bioinformatic analysis. FIG. 2C illustrates the portions of step 220 of the method 200, a method of preparing a genetic sample in a wet laboratory for bioinformatic analysis.

The method 200 can begin with sample collection. For example, the samples can be collected by receiving a nasal swab, blood, saliva, or other material potentially containing genetic material.

Accessioning Samples. Once received at the laboratory, at step 212, the samples can be accessioned, that is, prepared for later laboratory processes. For example, accessioning can include receiving a batch of samples. A batch of samples can include, for example, hundreds of individual samples, or thousands of individual samples. Each sample can be retained in a sample container. For example, test tubes can be used to store each of the samples. The sample containers can be sealed to help prevent environmental exposure and prevent sample co-mingling. For example, the sample containers may be sealed via a cap that is threaded, glued, press-fit, or otherwise affixed via appropriate sealing mechanism. When the samples are received in a batch, the corresponding sample containers may also include one or more remnants of a sampling tool, such as a swab used to collect the sample.

In some cases, the sample containers may be accompanied by Customer Sample Identifiers (CSI) such as by a component affixed to or integrated with the sample container. Such a CSI can uniquely distinguish individual sample containers from other sample containers being received. For example, a CSI may uniquely distinguish a sample from other samples in the same batch, other samples received on the same date, or other samples received from the same customer. Such CSI can be provided as a label such as a bar code or a Quick Response (QR) code, a chip such as a Radio Frequency Identifier (RFID), or another type of visual, transmission-generating, or other component affixed to or integrated with the sample container.

In some cases, the sample containers can be further sealed in an external container, such as a bag. External containers can help prevent contamination of samples, such as by preventing biological material from the samples contacting other or external surfaces. An external container can also help prevent cross-contamination between samples. Moreover, when a sample includes blood or other material, the external container can provide an additional barrier to protect technicians who may handle the samples. The external container can additionally include documentation correlating to the CSI, such as information on the patient that the sample was sourced from, information indicating circumstances of sampling, for example, a sampling date, a sampling method, a location that the sample was acquired, a name or title for a person who performed the sampling, other information, or combinations thereof.

In some cases, the samples can be in a chemical solution. For example, the sample may be prepared in an aqueous solution, such as a saline solution. In some cases, the samples can include a bodily fluid such as saliva, mucus, blood, or other. In an example, the sample can have a volume of about 2 mL, of about 3 mL, of about 4 mL, or of about 5 mL.

The samples include genetic material. For example, the samples can include Deoxyribonucleic Acid (DNA) or Ribonucleic Acid (RNA). In an example, the genetic material is one or more of many constituent components within the sample. For example, one portion of the genetic material may exist within the nuclei or mitochondria of white blood cells that are included within the sample. In another example, another portion of the genetic material may exist within viruses or bacteria within the sample. In these types of examples, the genetic material is not yet isolated from the remaining constituent components of the sample. Thus, the genetic material should be isolated.

To begin isolating the genetic material, batches of the samples can be heated in ovens to facilitate cell lysis. The temperature and duration of heating can be chosen such that any pathogenic material within the samples is rendered harmless, such that cellular lysis occurs, or both. For example, the samples can be heated at a temperature of between about 40° C. and 80° C., or at a temperature of between about 15° C. and 200° C., or at another appropriate temperature range. The samples can be heated for a time period of about 30 minutes, or for a time period of about 40 minutes, or for another appropriate time period. In some cases, such as where the samples are the contents of a blood draw, the heating step may be skipped.

After heating, the batches of samples can be removed from the ovens. In an example, sample containers can be removed from external containers, such as by cutting open the external containers. The sample containers can be inspected, either in a manual, automated, or semi-automated fashion. For example, a technician or an automated system can determine the CSI for the sample and compare the CSI to documentation accompanying the batch. If there is a discrepancy between the CSIs on the sample container and in the documentation, the sample may be flagged as having an error condition. Similarly, if the CSI on the sample container is damaged (such as by abrasion, heat-damage, or water-damage) and has become unreadable, the sample may be flagged as having an error condition.

In some cases, the technician or automated system can further inspect the contents of the sample container, such as visually. If the sample does not include expected constituent components, then the sample can be flagged as having an error condition. For example, if the sample includes a fluid that is not permitted (such as extraneous blood), includes an entire swab or no swab, is within a fractured or broken sample container, or is outside of an expected range of volume (e.g., between two and five milliliters), or other conditions, then the sample can be flagged as having an error condition.

Subsequently, samples that have not been flagged with an error condition can proceed to sample integration. Here, the sample can be assigned a Laboratory Sample Identifier (LSI). Such an LSI can uniquely identify the sample from other samples received in the same batch, received on the same day, processed in the same laboratory, handled by the same company for sequencing, or combinations thereof. The LSI can be stored in a laboratory sample database, and uniquely correlated to the CSI for the sample. The LSI can be associated with any error codes reported from the sample. Both the CSI and the LSI can both be applied to the sample container.

Sample Plating. Once accessioned, the samples can be plated at step 214. At this point, the sample have been successfully integrated into the laboratory environment and are ready for analytics. At this point, the samples can be prepared for transfer to a sample microplate. The sample microplate can be labeled with a unique identifier, which can distinguish the sample microplate from other sample microplates. For example, the sample microplate can be a solid body with about 40 wells to about 400 wells, distributed across rows and columns, each well having a capacity of about 30 μL to about 300 μL. In other examples, different size microplates with a different number of wells at varying volumes can be used.

The samples to be used on the microplate may be racked and the rack may be assigned an identifier, such as to allow a technician to understand which samples correspond to which LSIs. The technician may unseal the sample, such as by a manual, automated, or semi-automated tool to efficiently open the sample container. The tooling may, for example, unscrew, cut, or drill each sample container, to make the sample within available for physical transfer to the sample microplate.

The samples can then be transferred to the microplate, such as by an automated robot that operates an end effector in accordance with one or more programs for effective transfer of the samples. This can be done, for example, with a combination of actuators, piezoelectric elements, pressure systems, and/or other components operating the end effector of the robot. The end effector can uptake portions of the samples in micropipettes and transfer those samples to the corresponding wells in the microplate. In some cases, disposable tips can be used. In some cases, portions of the samples can be transferred. In some cases, reagents can be added to the samples. In some cases, controls can be included in the microplate. The sample microplate, once completed, can be transferred for further processing in the laboratory.

Sample Storage. After plating, the samples can be stored at step 216. In some cases, accessioned samples, plated samples, or other samples, are stored for later use. In this case, they can be stored at room temperature, or can be cryogenically frozen and arranged on racks for later retrieval. Samples can be preserved for periods of days or years to allow later rapid re-testing.

Extraction of Genetic Material. When genetic analysis is desired, the genetic material of the samples can be extracted for sequencing at step 222. In some examples, a reagent can be applied to sample wells to lyse cells therein to expose genetic material.

Additionally, aspirating, and dispensing reagents can be used to selectively bind genetic material released from lysed cells. In some examples, this can include applying a bead to the well. In this case, the beads can, for example, be magnetic beads that selectively bind to the genetic material. This can help allow for isolation and purification of the genetic material at the bead, leaving contaminants in the solution. In an example, a magnetic bead can be magnetically drawn to a magnetic base at or under the sample microplate. In this case, after the genetic material has been drawn to the bead, a flushing step can be performed to wash away remaining fluid, helping to remove impurities.

In some examples, fluid can be added or removed from wells, such as to concentrate or elute the genetic material. Fluid can be transferred from the wells of the sample microplate to a genome stock microplate. In an example, a portion of fluid can be removed from each well for quality control purposes. This can, for example, be used to determine concentration of genetic material therein.

Library Preparation. After extraction of the genetic material, a library can be prepared using the contents of the genome stock microplate at step 224. For example, the bead for each well, including ionically bonded genetic material, can be transferred to a distinct well of a library preparation microplate. The library preparation microplate can include an identifier. The LSI associated with each well on the sample microplate can be mapped to a corresponding well on the library preparation microplate. The library preparation microplate may be transferred to a new portion of the laboratory to help prevent amplified genetic material from entering portions of the laboratory where genetic material has not been amplified, which could result in contamination.

A reagent can be applied to each well of the library preparation microplate. The reagent can ionically bond to the surface of the bead within the well more strongly than the genetic material. This helps release the genetic material from the surface of the bead of each well, enabling the genetic material to be chemically interacted with.

Library preparation can include normalization of a concentration of genetic material in each well of the sample microplate. Library preparation can further include fragmentation of the genetic material via an enzyme or via the application of physical forces. During this process, the entire genome (e.g., roughly three billion base pairs for a human genome), may be fragmented into pieces. In an example, the pieces can be about 300 to 400 base pairs in length. These pieces can be referred to as nucleic acid fragments. These nucleic acid fragments can undergo adaptor ligation and indexing. In an example, this can include Next Generation Sequencing (NGS) library preparation processes.

The genetic material can then be amplified, such as by Polymerase Chain Reaction (PCR) amplification. The resulting solution can be purified and eluted. During this library preparation, one or more reference samples of genetic material can be added to the wells of the library preparation microplate. The reference samples can serve as controls and aid in quality control.

Once the library preparation has been completed, thousands or millions of distinct fragments of the genetic material, each corresponding with a different portion of a genome of the subject, can be ligated to predefined adapters that bind with the genetic material. Each of the adaptor ligated fragments is referred to as a “library.”

In additional examples, probes applied to each well can include chemical identifiers (“barcodes”) that are distinct from each other. The use of a different chemical identifier for probes applied to each well of the well plate can enable sequencing to later be performed for multiple subjects on the same flow cell, without conflating sequencing results for those subjects.

In additional examples, the library preparation process can further include controlling a concentration of the genetic material in each well, and purification and/or elution of the resulting material. Similar to the processes performed after extraction of genetic material, concentration of genetic material after library preparation can be confirmed for each well via testing.

Enrichment of Genetic Material. After library preparation, enrichment processes can be performed in order to either directly amplify (e.g., via amplicon or multiplexed PCR) or capture (e.g., via hybrid capture) predefined libraries of genetic material, such as at step 226 in FIG. 2C. This can enhance the ease of sequencing desired portions of the genome.

Here, desired assays or probes can be used during genetic sample enrichment, prior to amplification, to capture any targeted genetic material. The captured genetic material is amplified and sequenced. The sequenced genetic material is collected and called to produce a plurality of reads.

For example, during enrichment, customized biotinylated oligonucleotide probes can be applied to the libraries. The probes can selectively hybridize genetic material occupying desired portions of the genome for the genetic material, such as specific genes, or the entire exome. Magnetic beads can bind to biotin molecules in the probes to attach the hybridized material to the magnetic beads. Magnetic forces can capture the beads in place, enabling remaining fluid within each well to be removed or washed out, thereby removing impurities, and leaving only the genetic material that is desired. Thus, genetic material can be released from the beads in a similar manner to that discussed above for prior processes.

In an example, hybrid capture target enrichment can be performed. During this process, the probes can include tailored oligonucleotides that are chosen to bind to the genetic material. The range of probes can be tailored as a group to bind to specific alleles, specific genes, the exome, the entire genome, etc. That is, each probe can bind to a nucleic acid fragment at a specific location on the genome, and the range of probes can be selected to ensure that alleles, genes, the exome, or the entire genome of the subject being considered is acquired.

In these examples, utilizing probes in this manner can enhance efficiency of the sequencing process, by foregoing the need to sequence all of the roughly three billion base pairs found in the human genome. The enrichment process can further include controlling a concentration of the genetic material in each well, and purification and/or elution of the resulting material. Similar to the processes performed after extraction of genetic material, concentration of genetic material after enrichment can be confirmed for each well via testing.

Sequencing of Genetic Material. After enrichment, the genetic material can be sequenced at step 228. Sequencing can be performed according to any of a variety of techniques, including short-read and long-read techniques.

In an example, the sequencing can be performed as Sequencing by Synthesis (SBS) at genetic analyzer equipment. For example, sets of enriched libraries of genetic material bound to probes in earlier steps can be transferred to a flow cell, and annealed to oligonucleotide probes within the flow cell. At this stage, the contents of multiple wells can be applied to the same flow cell, because the libraries within those wells are tagged with the chemical identifiers referred to above.

In an example, the chemical identifiers can include nucleotide sequences that are detectable during the sequencing process to determine a corresponding LSI. Complementary sequences can then be created via enzymatic extension to create a double-stranded portion of genetic material. The double-stranded genetic material can then be denatured, and the library fragment can be washed away. Bridge amplification can then be performed to create copies of the remaining molecule in a localized cluster. For example, a cluster can comprise twenty to fifty copies of the same molecule, localized to a location the size smaller than a pinhead on the flow cell. Sequencing primers can be annealed to library adapters to prepare the flow cell for SBS. During SBS, the sequencing primer uses reverse terminator fluorescent oligonucleotides, one base per cycle, for several cycles in the forward direction. After the addition of each nucleotide, clusters can be excited by a light source, resulting in fluorescence which can be measured. The emission wavelength and signal intensity for each cluster determines a base call for that cluster. A chemical group blocking a 3′ end of the fragment can then be removed, enabling a subsequent nucleotide to be read. This can help control nucleotide addition and detection. After each cycle, denaturing and annealing can be performed to extend the index primer. A complementary reverse strand can be created and extended via bridge amplification. The reverse strand can then be read in the reverse direction for a number of cycles, in a manner similar to reads in the forward direction.

Depending on whether a complete human genome, or another set of genomic data, is being tested, different reagents can be chosen. That is, different reagents can be utilized for library preparation for a pathogen (e.g., bacteria, virus) or an organelle (e.g., mitochondria) than for a human genome, in addition to desired targeted DNA. Any targeted collected genetic material exhibiting Ribonucleic Acid (RNA) genomes can be translated to DNA before sequencing, enrichment, and/or library preparation are performed.

Throughout the processes discussed above, the laboratory environment can be carefully controlled to ensure quality. For example, temperature within each segment of the laboratory can be carefully monitored and controlled, and ultraviolet lighting or other features capable of inactivating genetic material can be carefully positioned to ensure that contamination does not occur.

In general, raw sequencing data generated during synthesis is stored in a file format such as Binary Base Call (BCL). This raw data may be fed to an analytical pipeline such as a cloud-based computing environment. Raw sequencing data may be processed by the pipeline into a second format, such as a text based FASTQ format, that reports quality scores. The second format is then analyzed to perform alignment of sequence reads to a reference genome, such as a reference genome reported in a Browser Extensible Data (BED) file. The aligned sequence data may be reported as a Binary Alignment Map (BAM) file.

The aligned sequence data may then be called, resulting in a Variant Call Format (VCF) file reporting called variants at each location of the genome that was sequenced, together with secondary metrics such as quality indicator metrics. The called sequence data may be provided to a data analyst via a User Interface (UI), such as a Graphical User Interface (GUI) presented via a display. The technician may then validate the resulting called sequence data and release it for reporting to subjects, health care providers, and/or scientists.

### Identity Verification for Genetic Files Using SNPs

Discussed herein is a method with two separate quality control filters for SNP-based identification (e.g., “fingerprinting”) of genetic material. The methods discussed herein are repeatable, reliable, and process efficient, without relying on metadata including personally identifiable information (PII) such as name, social security number, or other information. Examples of the methods discussed herein are depicted in FIGS. 3A and 3B.

FIG. 3A depicts a flow diagram of this process 300 for quality control and identification of VCFs. At first, the two VCFs can be received or collected at step 310. This can include identifying and retrieving a first VCF 312 and a second VCF 314. Each of the two VCFs being compared can then be analyzed to determine whether they belong to the same patient.

The first VCF 312 can be, for example, from an external source such as a third-party tool. For example, a third-party tool or program may process collected genetic data may comprise an analytical tool external to the company that is performing the quality control for the two VCFs. Such a third-party analytical tool may have been used to produce a VCF and corresponding CRAM file from genetic data (e.g., FASTQ-format). The first VCF 312 can, for example, not include metadata or other identifying data.

The second VCF 314 can be, for example, from an internal source. As used herein, “internal” may refer to being within a network owned and/or operated by the entity performing the quality control method, while “external” may refer to being separate from such internal components or operations (e.g., existing in a state that is out of the direct control or ambit of the entity performing the quality control). The second VCF 314 can be a premade VCF, associated with a particular individual. For example, the second VCF 314 can be made by an in-house analytical tool which performs a different analysis such as interpretation of exome data.

The second VCF 314 can be procured by a method using an identifier. For example, the operator can receive an identifier (ID) indicating a particular patient and associated VCF. The operator can retrieve the associated VCF and confirm the ID by comparing between two or more workflows in the internal system. The two separate workflows can be, for example, two sets of data, two cloud computing environments, or two sets of services, such as connected through links or other references.

The process 300 can, through a first quality control step 320 and a second quality control step 330, determine whether the first VCF 312 and the second VCF 314 are from the same individual, and help reassociate identifying information with the first VCF 312 processed by an external source.

The first quality control step 320 can include identifying SNPs within a predefined set (also referred to as a “predetermined set”) that meet a quality threshold. For example, predefined sets of 30 SNPs, 50 SNPs, 75 SNPs, 100 SNPs, or other amounts can be used depending on the specific analysis. The threshold can, for example, be a threshold read depth indicating a number of reads of the corresponding genomic coordinate, such as at least 20 reads, at least 30 reads, or at least 40 reads. The predefined set may comprise a set of SNPs, such as between twenty and five hundred SNPs, or between sixty and ninety SNPs (e.g., seventy-eight SNPs), and may comprise all or a subset of SNPs as described in Pengelly, R. J., Gibson, J., Andreoletti, G. et al. A SNP profiling panel for sample tracking in whole-exome sequencing studies. Genome Med 4, 89 (2013).

In one embodiment, the predefined set of SNPs comprises SNPs having high discriminatory power, even in large datasets. Examples, include SNPs located at least ten base pairs from exon boundaries, that are not situated in regions with a high sequence similarity to non-target regions (e.g., SNPs with a non-target BLAT score of <100), that are outside of linkage disequilibrium with all other SNPs in the predefined set, that represent bi-allelic distributions of non-complementary bases, that are not present in large-scale genomic repeats, and/or that are located within regions having GC content between 40% and 45%.

In a further embodiment, the predefined set of single nucleotide polymorphisms are selected based on their prevalence in a target area of analysis. For example, a single nucleotide polymorphism can be prevalent in the target area if it is commonly found in that target area. In some cases, such a target area of analysis can be a particular portion of genetic code or material, such as a portion of genetic code that can indicate a particular phenotype, particular condition, or particular disease. The predetermined set of single nucleotide polymorphisms can, for example, be ones found commonly in such a target area, e.g., having high prevalence. In another example, the predetermined set of single nucleotide polymorphisms can be ones of low prevalence in the target area. The predetermined set of single nucleotide polymorphisms can be changed depending on the specific target area and desired analysis.

The quality threshold may be defined as a quality score. A quality score can be used to indicate a confidence level associated with the call for a SNP, based on the consistency (e.g., concordance) of individual reads for that SNP. For example, a Phred quality score of at least twenty (e.g., between twenty and sixty) can be used as a quality threshold for a SNP, which would indicate 99% confidence in the call for that SNP (e.g., as reported within a corresponding VCF).

If less than a minimum number of SNPs in the predefined set (e.g., less than half of the SNPs in the predefined set, less than two-thirds of the SNPs in the predefined set, etc.) meet the quality threshold, then the two VCFs lack data of sufficient quality to confirm that the VCFs match. However, if at least a minimum percentage of SNPs in the predefined set meet the quality threshold, then the process can move to the second step.

In one embodiment, it is insufficient for each VCF to have at least the minimum number of SNPs of the predefined set meeting the quality threshold. Rather, for a SNP of the predefined set to be counted towards the minimum number, it must meet the quality threshold within both the first VCF 312 and the second VCF 314. That is, information at a corresponding genomic loci for the SNP, for both VCFs, should meet the quality threshold. In short, a SNP from the predefined set must be reported by both VCFs and meet the quality threshold within both VCFs in order to be counted towards the minimum number.

In a further example, at quality control step 320, both VCFs can be checked to determine whether each includes at least half of the SNPs from the predefined set at the quality threshold. According to this example, if half or more of SNPs in the predefined set are present in both of the VCFs and meet the quality threshold within both of the VCFs, the first quality control step 320 is passed at step 324. However, if less than half of the predetermined set of SNPs are found to meet the quality threshold in one or both of the VCFs, then the quality control step 320 reports/flags the VCFs in memory as unable to be confirmed as belonging to the same patient at step 322.

The second quality control step 330 can include comparing SNPs in the predefined set that meet the desired quality threshold at corresponding genomic loci across both VCFs. The SNPs in the predefined set that meet the desired quality threshold are hereinafter referred to as “qualified SNPs”. Here, if at least a threshold amount of the calls of qualified SNPs at corresponding loci match between the two VCFs, then the two VCFs are confirmed as referring to the same individual. As used herein, a threshold amount may comprise a number or percentage, such a percentage determined by evaluating genotype concordance. A match may comprise calls for the same variant at the genomic locus/loci for a SNP within both of the VCFs.

For example, at quality control step 330 genotype concordance can be computed over all qualified SNPs for both VCFs. The amount of genotype concordance of qualified SNPs between the two VCFs can be calculated. The calculated amount can be filtered at step 340 based on whether or not the calculated amount is greater than or less than a threshold amount. If there is a large genotype concordance, such as equal to or above a threshold amount of 95%, the quality control step 330 can pass at step 342. However, if less than the threshold amount such as less than 95%, the quality control step 330 report/flags the VCFs in memory the VCFs as unable to be confirmed as belonging to the same patient at step 344.

FIG. 3B shows a closer look at second quality control step 330 of the process 300. Here, the first VCF 312 can be obtained from an external source, and the second VCF 314 can be obtained from an internal source. Once the first and second VCFs have passed the first quality control step 320 as discussed above, they can be compared at the second quality control step 330.

The threshold amount can be calculated to a desired amount, for example, 90%, about or above 90, 91, 92, 93, 94, 95, 96, 97, 98, or 99%, depending on the specific SNPs selected for the predefined set, historical data, or other analytical data, which may be tailored to the specific genetic samples being analyzed and quality controlled.

If the threshold amount is met, the matching identity of the first VCF and the second VCF can be confirmed at 313. If the threshold amount is not met, the matching identity of the first VCF and the second VCF is not confirmed at 315, and the quality control process 300 reports/flags the VCFs in memory as unable to be confirmed as belonging to the same patient. Thus, the first VCF 312 and the second VCF 314 are not confirmed as having the same origin. Alternatively, if the first VCF and the second VCF are indeed confirmed to be from the same individual, the first VCF 312 can be reassociated with identifying information, and used in other genetic analyses.

### Computer Examples

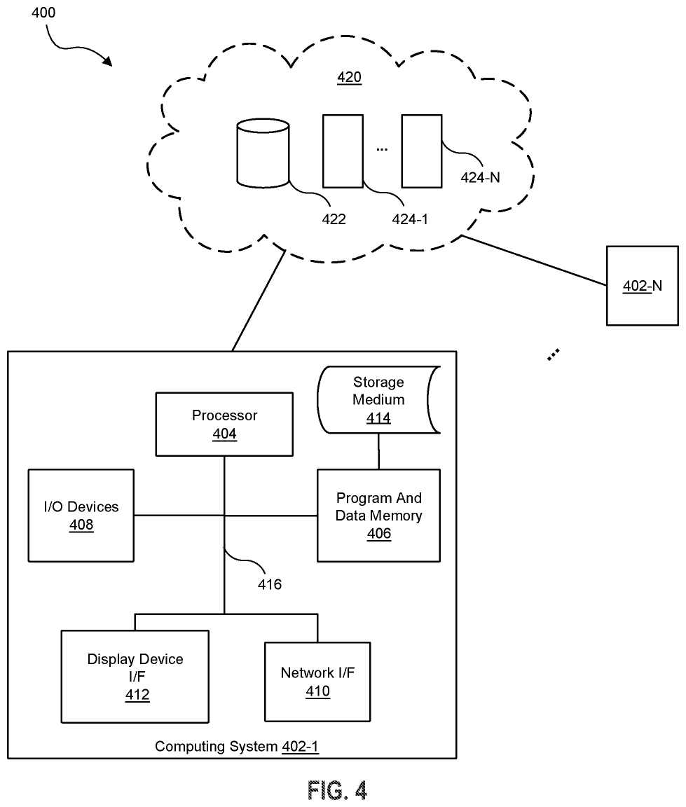

FIG. 4 depicts an example cloud computing system 400 operable to perform the above methods by executing programmed instructions tangibly embodied on one or more computer readable storage mediums. The cloud computing system 400 generally includes the use of a network of remote servers hosted on the internet to store, manage, and process data, rather than a local server or a personal computer (e.g., in the computing systems 402-1-402-N). Cloud computing enables users to use infrastructure and applications via the internet, without installing and maintaining them on-premises. In this regard, the cloud computing network 420 may include virtualized information technology (IT) infrastructure (e.g., servers 424-1-424-N, the data storage module 422, operating system software, networking, and other infrastructure) that is abstracted so that the infrastructure can be pooled and/or divided irrespective of physical hardware boundaries. In some embodiments, the cloud computing network 420 can provide users with services in the form of building blocks that can be used to create and deploy various types of applications in the cloud on a metered basis.

Various components of the cloud computing system 400 may be operable to implement the above operations in their entirety or contribute to the operations in part. Some embodiments disclosed herein may utilize instructions (e.g., code/software) accessible via a computer-readable storage medium for use by various components in the cloud computing system 400 to implement all or parts of the various operations disclosed hereinabove. Examples of such components include the computing systems 402-1-402-N.

Example components of the computing systems 402-1-402-N may include at least one processor 404, a computer readable storage medium 414, program and data memory 406, input/output (I/O) devices 408, a display device interface 412, and a network interface 410. For the purposes of this description, the computer readable storage medium 414 comprises any physical media that is capable of storing a program for use by one or more of the computing systems 402.-1-402-N. For example, the computer-readable storage medium 414 may be an electronic, magnetic, optical, electromagnetic, infrared, semiconductor device, or other non-transitory medium. Examples of the computer-readable storage medium 414 include a solid-state memory, a magnetic tape, a removable computer diskette, a random-access memory (RAM), a read-only memory (ROM), a rigid magnetic disk, and an optical disk. Some examples of optical disks include Compact Disk-Read Only Memory (CD-ROM), Compact Disk-Read/Write (CD-R/W), Digital Versatile Disc (DVD), and Blu-Ray Disc.

The processor 404 is coupled to the program and data memory 406 through a system bus 416. The program and data memory 406 include local memory employed during actual execution of the program code, bulk storage, and/or cache memories that provide temporary storage of at least some program code and/or data in order to reduce the number of times the code and/or data are retrieved from bulk storage (e.g., a hard disk drive, a solid state drive, or the like) during execution.

Input/output or I/O devices 408 (including but not limited to keyboards, displays, touchscreens, microphones, pointing devices, etc.) may be coupled either directly or through intervening I/O controllers. Network adapter interfaces 410 may also be integrated with the system to enable the computing system 402 to become coupled to other computing systems or storage devices through intervening private or public networks. The network adapter interfaces 410 may be implemented as modems, cable modems, Small Computer System Interface (SCSI) devices, Fibre Channel devices, Ethernet cards, wireless adapters, etc. Display device interface 412 may be integrated with the system to interface to one or more display devices, such as screens for presentation of data generated by the processor 404.

The devices described herein may be configured to include computer-readable non-transitory media storing computer readable instructions and one or more processors coupled to the memory, and when executing the computer readable instructions configure the cloud computing system 400 to perform method steps and operations described above. The computer-readable non-transitory media includes all types of computer readable media, including magnetic storage media, optical storage media, flash media and solid-state storage media.

Software including one or more computer-executable instructions that facilitate processing and operations as described above with reference to any one or all of steps of the disclosure may be installed in and sold with one or more servers and/or one or more routers and/or one or more devices within consumer and/or producer domains consistent with the disclosure. Alternatively, the software may be obtained and loaded into one or more servers and/or one or more routers and/or one or more devices within consumer and/or producer domains consistent with the disclosure, including obtaining the software through physical medium or distribution system, including, for example, from a server owned by the software creator or from a server not owned but used by the software creator. The software may be stored on a server for distribution over the Internet, for example.

Also, it will be understood by one skilled in the art that this disclosure is not limited in its application to the details of construction and the arrangement of components set forth in the description or illustrated in the drawings. The examples herein are capable of other examples, and capable of being practiced or carried out in various ways.

The phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting.

The components of the illustrative devices, systems and methods employed in accordance with the illustrated examples may be implemented, at least in part, in digital electronic circuitry, analog electronic circuitry, or in computer hardware, firmware, software, or in combinations of them. These components may be implemented, for example, as a computing program product such as a computing program, program code or computer instructions tangibly embodied in an information carrier, or in a machine-readable storage device, for execution by, or to control the operation of, data processing apparatus such as a programmable processor, a computer, or multiple computers.

A computing program may be written in any form of programming language, including compiled or interpreted languages, and it may be deployed in any form, including as a stand-alone program or as a module, component, subroutine, or other unit suitable for use in a computing environment. A computing program may be deployed to be executed on one computer or on multiple computers at one site or distributed across multiple sites and interconnected by a communication network. Also, functional programs, codes, and code segments for accomplishing the techniques described herein may be easily construed as within the scope of the present disclosure by programmers skilled in the art. Method steps associated with the illustrative examples may be performed by one or more programmable processors executing a computing program, code or instructions to perform functions (e.g., by operating on input data and/or generating an output). Method steps may also be performed by, and apparatus may be implemented as, special purpose logic circuitry, e.g., an FPGA (field programmable gate array) or an ASIC (application-specific integrated circuit), for example.

The various illustrative logical blocks, modules, and circuits described in connection with the examples disclosed herein may be implemented or performed with a general-purpose processor, a digital signal processor (DSP), an ASIC, a FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computing program include, by way of example, both general and special purpose microprocessors, and any one or more processors of any kind of digital computer. Generally, a processor will receive instructions and data from a read-only memory or a random-access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computing program instructions and data include all forms of non-volatile memory, including by way of example, semiconductor memory devices, e.g., electrically programmable read-only memory or ROM (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory may be supplemented by or incorporated in special purpose logic circuitry.

Those of skill in the art understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

Those of skill in the art further appreciate that the various illustrative logical blocks, modules, circuits, and algorithm steps described in connection with the examples disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the disclosure. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor may read information from, and write information to, the storage medium. In the alternative, the storage medium may be integral to the processor. In other words, the processor and the storage medium may reside in an integrated circuit or be implemented as discrete components.

As used herein, “machine-readable medium” means a device able to store instructions and data temporarily or permanently and may include, but is not limited to, random-access memory (RAM), read-only memory (ROM), buffer memory, flash memory, optical media, magnetic media, cache memory, other types of storage (e.g., Erasable Programmable Read-Only Memory (EEPROM)), and/or any suitable combination thereof. The term “machine-readable medium” should be taken to include a single medium or multiple media (e.g., a centralized or distributed database, or associated caches and servers) able to store processor instructions. The term “machine-readable medium” shall also be taken to include any medium, or combination of multiple media, that is capable of storing instructions for execution by one or more processors, such that the instructions, when executed by one or more processors cause the one or more processors to perform any one or more of the methodologies described herein. Accordingly, a “machine-readable medium” refers to a single storage apparatus or device, as well as “cloud-based” storage systems or storage networks that include multiple storage apparatus or devices. The term “machine-readable medium” as used herein excludes signals per se.

### EXAMPLES

Various examples of the present disclosure can be better understood by reference to the following Examples which are offered by way of illustration. The present disclosure is not limited to the Examples given herein.

### Example 1. Identify Confirmation with Comparison of Third Party VCF to Internal VCF Using SNPs

An example quality control method was run on two VCFs. Genetic data was collected and recorded according to standard procedures, such as described with reference to FIGS. 2A to 2C above. The genetic data was received by both a third-party analytical tool and an internal tool.

A method similar to those discussed above was used to test the sample. The method is summarized in Table 1 below:

TABLE 1
Sample Method.

| Step | Parameters | Category |
|---|---|---|
| 1 | Receive ID | File procurement |
| 2 | Locate First VCF (third-party) | File procurement |
| 3 | Retrieve Second VCF (internal) | File procurement |
| 4 | Confirm ID associated with the | File procurement |
| | Second VCF (internal) | |
| 5 | Confirm Second VCF (internal) | File procurement |
| 6 | Select first VCF and second VCF | File processing |
| 7 | Read and validate file formats | File processing |
| | of first VCF and second VCF | |
| 8 | Retrieve predetermined set | First Quality Control Step |
| | of SNPs | |
| 9 | Determine overlapping SNPs | First Quality Control Step |
| 10 | Determine if data is sufficient | First Quality Control Step |
| | to move to QC step 2 | |
| 11 | Calculate genotype concordance | Second Quality Control Step |
| | score | |
| 12 | Determine if genotype concordance | Second Quality Control Step |
| | score is sufficient | |
| 13 | Determine if first VCF and second | Second Quality Control Step |
| | VCF originate from the same ID | |

First, (step 1) the identification (ID) of the workflow to be verified was received by the system. This ID indicated two VCFs that were to be obtained and compared for quality-control purposes using the method discussed herein, such as to verify that both VCFs originated from the same patient.

Second, (step 2) the first VCF was then received from the third-party tool, DRAGEN Secondary Analysis tool from Illumina in this Example. Initially, a compressed file was located from running the DRAGEN Secondary Analysis tool. The third-party tool received the genetic data in FASTQ-format. To ensure privacy, the genetic data received by the third-party did not include identifying information. This third-party tool was used to provide both the first VCF and a CRAM file (e.g., alignment information).

Third, (step 3) the second VCF was procured internally simultaneously to receipt of the first VCF from the DRAGEN Secondary Analysis tool. For procurement of the second VCF, an in-house analytical tool was used to interpret exome data and produce the second VCF. Because this tool was in-house, identifying information in the form of a reference ID was associated with this second VCF.

In this Example, the reference ID was inputted into an internal query database service storing the mapping between different workflows to get the ID for the reference workflow. Here, the latest usable ID of the reference workflow was selected (step 4). The variant file for the reference ID was looked for in the output bucket of the internal processing tool (step 4).

At this point (step 6), both the first VCF (from the third-party tool) and the second VCF (from an internal source) were selected and ready to be matched, e.g., compressed and indexed, in order to validate and compare them. The file formats were read and validated (step 7).

The first VCF and the second VCF were subjected to the two-step quality control analysis discussed above with reference to FIG. 3A and FIG. 3B. First, a predefined set of seventy-nine SNPs was selected (step 8). The DRAGEN Secondary Analysis tool from Illumina was used to generate calls for these predetermined SNPs for the first VCF.

At the first quality control step, the first VCF and the second VCF were analyzed to see if they each provided a threshold amount of the predefined set of SNPs meeting a quality threshold corresponding with a Phred quality score of 20 (step 9). At this first quality control step, data for the set of SNPs (e.g., variants) across both the first VCF and the second VCF were reviewed at the genomic locations of the predefined set of SNPs. At this step, it was determined whether too much data was missing to continue the comparison (i.e., if fewer than the threshold amount of SNPs met the quality threshold within both of the VCFs), or if enough data was present to continue the comparison (step 10). If the length of the set of SNPs meeting the quality threshold was too short, e.g., if less than half of the SNPs in the predefined set met the quality threshold, the two files were not comparable and were not subject to the second quality control step, as too much data was missing. Here, the threshold amount was set at 40% of the predefined set of SNPs. In this example, both the first VCF and the second VCF met this threshold. Those SNPs of the predefined set meeting the quality threshold were identified as qualified SNPs.

At the second quality control step, the first VCF and the second VCF were compared at the corresponding genomic loci of the qualified SNPs. A genotype concordance score was calculated for the qualified SNPs of the VCFs (step 11): the genotype concordance score was calculated as the number of qualified SNPs having matching calls (e.g., calls for the same nucleotide) at the same genomic loci between the first VCF and the second VCF, divided by the total number of qualified SNPs. In another example, this can be calculated by the number of matches of qualifying SNPs divided by the total number of qualifying SNPs.

A threshold amount of 95% was set. If the concordance score was at or above the threshold amount of 95%, the first VCF and the second VCF were determined to be from the same patient (step 12).

Here, when the first VCF and the second VCF were subject to the second quality control step, a 99% genotype concordance was confirmed (step 13). Thus, the first VCF and the second VCF were confirmed as reciting sequencing data for the same patient, and the first VCF was reassociated with identifying information.

Additional test cases were run, summarized in Table 1 below:

TABLE 1
Test Cases.

| Test Case | Parameters | Results |
|---|---|---|
| 0 | Regular | Success |
| 1 | No First VCF (third party) (step 2) | Fail |
| 2 | No Second VCF (internal) (step 3) | Fail |
| 3 | VCF missing header (step 7) | Fail |
| 4 | Low quality VCF (step 10) | Fail |
| 5 | First VCF and second VCF from different | Fail |
| | individuals (step 12) | |
| 6 | VCF genotype manually modified at a variant | Success |
| | location (step 13) | |
| 7 | VCF modified to remove a variant location | Success |
| | (step 13) | |
| 8 | VCF from the same individual produced from | Success |
| | different individuals (step 13) | |

In Test Case 1 the method was run without a third-party produced VCF file (first VCF), and the method failed to confirm VCFs as referring to the same patient. This indicated that the method functioned as desired. In Test Case 2, the method was run without an internally produced VCF file (second VCF), and the method failed to confirm VCFs as referring to the same patient. This indicated that the method functioned as desired. In Test Case 3, the use of a compressed VCF file without a header failed when the files were confirmed prior to the quality control steps. This indicated that the method functioned as desired.

In Test Case 4, one of the two VCF files was modified to include only quality scores below the quality threshold, and it failed during the first quality control step; the method indicated too much data was missing to proceed. This indicated that the method functioned as desired.

In Test Case 4, the two VCF files were taken from different individuals. The method failed at the second quality control step, indicating that the genotype concordance did not meet the threshold amount. This indicated that the method functioned as desired.

In Test Case 6, the two VCF files originated from the same individual, but one of the VCF files was manually changed to alter the genotype at one of the predetermined variant locations. The concordance score was still about 98%.

In Test Case 7, the two VCF files were from the same individual, but one VCF was manually modified to remove a SNP from the predefined set. A score of 1 was received at step 13, corresponding with success in confirming that the VCFs belonged to the same individual.

In Test Case 8, the two VCF files were from the same individual, but intentionally mislabeled as being produced from different individuals. A score of 1 was received at step 13, corresponding with success in confirming that the VCFs belonged to the same individual.

By comparison, in Test Case 0, where the methodology, such as discussed above with reference to FIGS. 3A-3B and Table 1, was followed, success was reached, and the first VCF (third party) and the second VCF (internal) were successfully matched as being from the same individual.

The terms and expressions that have been employed are used as terms of description and not of limitation, and there is no intention in the use of such terms and expressions of excluding any equivalents of the features shown and described or portions thereof, but it is recognized that various modifications are possible within the scope of the examples of the present disclosure. Thus, it should be understood that although the present disclosure has been specifically disclosed by specific examples and optional features, modification and variation of the concepts herein disclosed may be resorted to by those of ordinary skill in the art, and that such modifications and variations are considered to be within the scope of examples of the present disclosure.

### Additional Examples

In some aspects, the techniques described herein relate to a quality control method including: receiving a first variant call file; comparing the first variant call file to a second variant call file across a predetermined set of single nucleotide polymorphisms; and determining whether the first variant call file and the second variant call file originate from the same patient by determining whether at least a threshold amount of single nucleotide polymorphisms within the predetermined set match between the first variant call file and the second variant call file.

In some aspects, the techniques described herein relate to a quality control method including: receiving a first variant call file; comparing the first variant call file to a second variant call file across a predetermined set of single nucleotide polymorphisms; and determining whether the first variant call file and the second variant call file originate from the same individual by determining whether at least a threshold amount of single nucleotide polymorphisms within the predetermined set match between the first variant call file and the second variant call file.

In some aspects, the techniques described herein relate to a method, further including determining that at least half of the predetermined set of single nucleotide polymorphisms are called in both the first variant call file and the second variant call file prior to determining whether at least a threshold amount of single nucleotide polymorphisms within the predetermined set match.

In some aspects, the techniques described herein relate to a method, further including receiving genetic information from a third party and processing the genetic information to produce the first variant call file.

In some aspects, the techniques described herein relate to a method, further including producing a compressed reference-oriented alignment map file corresponding to the first variant call file.

In some aspects, the techniques described herein relate to a method, further including receiving the second variant call file from an internal source.

In some aspects, the techniques described herein relate to a method, wherein receiving the second variant call file from an internal source includes receiving an identifier correlating to the second variant call file and using the identifier to confirm the second variant call file across two different workflows.

In some aspects, the techniques described herein relate to a method, further including determining that the first variant call file and the second variant call file originate from the same individual when the first variant call file and the second variant call file match at a threshold amount of the predetermined single nucleotide polymorphisms.

In some aspects, the techniques described herein relate to a method, wherein the threshold amount of the predetermined single nucleotide polymorphisms includes 95% of the set.

In some aspects, the techniques described herein relate to a method, wherein the predetermined single nucleotide polymorphisms includes 20 single nucleotide polymorphisms.

In some aspects, the techniques described herein relate to a method, wherein the predetermined single nucleotide polymorphisms includes 100 single nucleotide polymorphisms.

In some aspects, the techniques described herein relate to a method, wherein the predetermined single nucleotide polymorphisms includes 300 single nucleotide polymorphisms.

In some aspects, the techniques described herein relate to a method, further including determining that the first variant call file and the second variant call file originate from the same patient when the first variant call file and the second variant call file match at the threshold amount of the predetermined single nucleotide polymorphisms having more than a predetermined read depth.

In some aspects, the techniques described herein relate to a method, wherein the predetermined read depth includes 20 reads.

In some aspects, the techniques described herein relate to a method, further including producing a confidence rating based on comparing the first variant call file and the second variant call file at the predetermined single nucleotide polymorphisms.

In some aspects, the techniques described herein relate to a method, further including determining that the first variant call file and the second variant call file originate from the same patient if the confidence of a corresponding call is 99% or greater.

In some aspects, the techniques described herein relate to a method, further including selecting the predetermined set of single nucleotide polymorphisms.

In some aspects, the techniques described herein relate to a method, wherein selecting the predetermined set of single nucleotide polymorphisms includes selecting the single nucleotide polymorphisms from a database.

In some aspects, the techniques described herein relate to a quality control method including: receiving a first variant call file from an external source; receiving a second variant call file; comparing the first variant call file to the second variant call file at a predetermined number of single nucleotide polymorphisms; and determining whether the first variant call file and the second variant call file are from the same individual based on comparing at the predetermined single nucleotide polymorphisms.

In some aspects, the techniques described herein relate to a method, wherein comparing at the predetermined single nucleotide polymorphisms includes checking that at least half of the single nucleotide polymorphisms are in both the first variant call file and the second variant call file.

In some aspects, the techniques described herein relate to a method, wherein comparing at the predetermined single nucleotide polymorphisms includes computing genotype concordance over the predetermined single nucleotide polymorphisms.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A quality control method comprising:
receiving a first variant call file;
comparing the first variant call file to a second variant call file across a predetermined set of single nucleotide polymorphisms; and
determining whether the first variant call file and the second variant call file originate from a same individual by determining whether at least a threshold amount of single nucleotide polymorphisms within the predetermined set match between the first variant call file and the second variant call file.

2. The method of claim 1, further comprising determining that at least half of the predetermined set of single nucleotide polymorphisms are called in both the first variant call file and the second variant call file prior to determining whether at least a threshold amount of single nucleotide polymorphisms within the predetermined set match.

3. The method of claim 1, further comprising receiving genetic information from a third party and processing the genetic information to produce the first variant call file.

4. The method of claim 1, further comprising selecting the single nucleotide polymorphisms in the predetermined set according to one or more criteria comprising prevalence of the single nucleotide polymorphisms in a target region, and selecting a minimum number of single nucleotide polymorphisms according to the one or more criteria; and wherein determining whether at least the threshold amount of single nucleotide polymorphisms within the predetermined set match between the first variant call file and the second variant call file comprises calculating a quality score based on the single nucleotide polymorphisms within the predetermined set.

5. The method of claim 1, wherein receiving the second variant call file comprises receiving an identifier correlating to the second variant call file and using the identifier to confirm the second variant call file across two different workflows.

6. The method of claim 1, further comprising determining that the first variant call file and the second variant call file originate from the same individual when the first variant call file and the second variant call file match at a threshold amount of the predetermined single nucleotide polymorphisms.

7. The method of claim 1, wherein the threshold amount of the predetermined single nucleotide polymorphisms comprises 95% of the predetermined set.

8. The method of claim 7, wherein the predetermined single nucleotide polymorphisms comprises 20 single nucleotide polymorphisms.

9. The method of claim 8, wherein the predetermined single nucleotide polymorphisms comprises 100 single nucleotide polymorphisms.

10. The method of claim 1, wherein the predetermined single nucleotide polymorphisms are selected based on their prevalence in a target area of analysis.

11. The method of claim 1, wherein determining whether at least a threshold amount of single nucleotide polymorphisms within the predetermined set match between the first variant call file and the second variant call file comprises confirming that a minimum percentage of the predetermined set meet a quality threshold corresponding with a threshold read depth.

12. The method of claim 11, wherein the quality threshold comprises a quality score based on concordance of individual reads from the predetermined set.

13. The method of claim 1, further comprising producing a confidence rating based on comparing the first variant call file and the second variant call file at the predetermined single nucleotide polymorphisms.

14. The method of claim 1, further comprising determining that the first variant call file and the second variant call file originate from the same individual if confidence of a corresponding call is 99% or greater.

15. The method of claim 1, further comprising selecting the predetermined set of single nucleotide polymorphisms.

16. A quality control method comprising:
receiving a first variant call file from an external source;
receiving a second variant call file;
comparing the first variant call file to the second variant call file at a predetermined number of single nucleotide polymorphisms; and
determining whether the first variant call file and the second variant call file are from a same individual based on comparing at the predetermined single nucleotide polymorphisms.

17. The method of claim 16, wherein comparing at the predetermined single nucleotide polymorphisms comprises checking that at least half of the single nucleotide polymorphisms are in both the first variant call file and the second variant call file.

18. The method of claim 16, wherein comparing at the predetermined single nucleotide polymorphisms comprises computing genotype concordance over the predetermined single nucleotide polymorphisms.

19. A quality control method comprising:
selecting a first Variant Call File (VCF);
selecting a second VCF;
identifying a predetermined set of Single Nucleotide Polymorphisms (SNPs) for comparison;
determining an amount of qualified SNPs of the predetermined set within the VCF and the second VCF, each qualified SNP meeting a quality threshold indicating sequencing quality;
in an event that the amount of qualified SNPs is less than a minimum number, flagging the first VCF and the second VCF as unable to be confirmed as belonging to a same individual;
in an event that the amount of qualified SNPs is equal to or greater than the minimum number:
determining an amount of the qualified SNPs that have the same call within both the first VCF and the second VCF;
in an event that the amount of the qualified SNPs that have the same call within both the first VCF and the second VCF is less than a threshold amount, flagging the first VCF and the second VCF as unable to be confirmed as belonging to the same individual; and
in an event that the amount of the qualified SNPs that have a same call within both the first VCF and the second VCF is greater than or equal to the threshold amount, flagging the first VCF and the second VCF as belonging to the same individual.

20. The method of claim 19 wherein:
the predetermined set of SNPs comprise between twenty and five hundred SNPs that are each located at least ten base pairs from an exon boundary;
the quality threshold comprises a Phred quality score of at least twenty;
the minimum number comprises at least half of the predetermined set of SNPs; and
the threshold amount is at least ninety-five percent.
